(19) United States
(12) Patent Application Publication
(10) Pub. No.: US 2006/0263327 A1
(43) Pub. Date: Nov. 23, 2006

(54) Method for the production of a stable injectable formulation made of difficult to dissolve antineoplastic active substances

(76) Inventors: Gerhard Eidenhammer, Mondsee (AT); Heinz Schnait, Seewalchen (AT)

Correspondence Address: WENDEROTH, LIND & PONACK, L.L.P., 2033 K STREET N. W., SUITE 800, WASHINGTON, DC, 20006-1021, US

(21) Appl. No.: 10/540,254

(22) PCT Filed: May 17, 2004
(86) PCT No.: PCT/EP04/05268

§ 371 Date: Aug. 17, 2005

(30) Foreign Application Priority Data
Mar. 19, 2003 (AT) A 7602003

(51) Int. Cl. A61K 31/785; A61K 31/4745; A61K 31/337; A61K 36/47

(52) U.S. Cl. 424/78.12; 514/283; 514/449; 424/731

(57) ABSTRACT

The invention relates to a method for the production of a stable injectable formulation of antineoplastic active substances which are difficult to dissolve. A formulation containing the antineoplastic active substance and a solvent and/or a solvent system containing, optionally, a solubilisation agent is treated with a cation exchanger.

Paclitaxel or Taxol is obtained for example from the bark of the pacific yew tree, taxus brevifolia, and represents an agent by now widely established as an antineoplastic drug.

Treatment with paclitaxel is for example described in the case of ovarian cancer (cf. inter alia McGuire et al. Ann. Int. Med., 111, 273-279), breast cancer (cf. inter alia Holmes et al., Proceedings of the American Society of Clinical Oncology, Vol. 10, pp. 60), lung cancer (cf. inter alia Brown et al., J of Clin Oncol, Vol. 9, No. 7, pp. 1261-1267), leukemia (cf. inter alia Rowinsky et al., Cancer Research 49, 4640-4647).

Paclitaxel itself is only poorly soluble in water. It is therefore necessary to employ a solvent for the preparation of, for example, injectable formulations of paclitaxel. It should also be possible to dilute these formulations further with aqueous infusion solutions (for example 0.9% NaCl solution, 5% glucose). However, paclitaxel is insufficiently stable in lipophilic solvents, for example polyoxyethylene castor oil, such as Cremophor EL® or Cremophor ELP®, as well as also in alcoholic solvents, such as for example ethanol, and breaks down when stored at ambient temperature.

Paclitaxel itself is insufficiently stable in such solutions and undergoes degradation.

WO 94/12030 discloses a formulation of paclitaxel in polyoxyethylene castor oil (cremophor), which has a pH value of less than 8.1 adjusted by adding organic or inorganic acids, preferably by adding citric acid.

In EP 0 654 145 a stabilized formulation of a pharmaceutical compound, such as for example paclitaxel, is described, which contains a solvent and a nonionic solubilizing agent, the formulation having a sufficiently low carboxylate ion content in order to prevent the degradation of the paclitaxel. The adjustment of the pH value to below 8.1 takes place by adding HBr, HF or HI.

WO 98/33780 describes formulations of paclitaxel with purified cremophor, which has a certain maximum content of at least one certain cation.

WO 97/12017 discloses a method for purifying cremophor, wherein aluminum oxides or silicates are utilized for the purification. Such purified cremophors are employed for the production of drug preparations, especially also for the production of paclitaxel formulations.

The intention in all of these methods is ensuring a satisfactory stability of the formulation by treating the solubilizing agent and/or by adjusting a certain pH value of the solution of paclitaxel. However, the stability of such formulations continues to be unsatisfactory.

The aim of the invention is therefore to provide a stable injectable formulation of paclitaxel, which has improved stability and can be produced in simple manner.

Subject matter of the invention is therefore a method for the production of a stable injectable formulation of antineoplastic agents, characterized in that a formulation comprising the antineoplastic agent and a solvent and/or solvent system, which optionally contains a solubilizing agent, is treated with a cation exchanger.

The formulations according to the invention have improved stability.

The concentration of the active agent in the formulation can be 1-10 mg/ml, preferably 2-8 mg/ml, highly preferably 6 mg/ml.

As the solvent or solvent systems with solubilizing agents are considered for example ethanol, ethanol/polyoxyethylene castor oil, ethanol/polysorbate, ethanol/polyethylene glycol and the like, where the mixing proportions may vary between 90:10 and 30:70 depending on the system.

In the solvent system ethanol/polyoxyethylene castor oil the fraction of ethanol is preferably 90-50 parts. In the solvent system ethanol/polysorbate the fraction of ethanol is preferably 40-60 parts.

The active agent is dissolved in a solvent or solvent system and the solution is stirred at ambient temperature until the paclitaxel is completely dissolved. The solution is subsequently treated with a cation exchanger.

Cation exchangers to be considered are those containing sulfonic acid groups or carboxylate groups.

Cation exchangers that are utilizable can contain a matrix of polystyrene, polystyrene divinyl benzene copolymer, polyacrylic ester, methacrylic ester divinyl benzene copolymer, cellulose (for example Amberlite®, Amberlyst®, Dowex®). The activity is typically between 2.0-4.0 meq/g.

The quantity of the cation exchanger is preferably 0.01-10% of the total quantity of the batch, preferably 0.05-1%. The length of treatment is typically between 2 and 24 hours.

The solution treated in this manner is subsequently filtered through a filter of appropriate pore size, which permits the complete separation of the cation exchangers, and filled directly into vials.

Prepurification of the solvent or the solvent system is not required. Thereby no additional equipment is necessary for the distillation of the polyoxyethylene castor oil and/or the filtration over columns before or after the treatment with a cation exchanger if, for example, a solvent system containing polyoxyethylene castor oil is utilized.

In order to test their stability, formulations or comparison solutions prepared in this manner were stored for 2 weeks at 60° C. The content of degradation products and the content of the active agent in the solution were subsequently determined.

It was found that the active agent content was markedly higher than in the comparison samples, which had either not been treated or had been stabilized with additives. Further, the content of known decomposition products as well as the sum of the decomposition products were markedly lower than was the case in the comparison samples.

### EXAMPLES

### Example 1 and 2

Polyoxyethylene castor oil (12.72 g) is dissolved in ethanol (87.82 g). The solution is stirred until all of the polyoxyethylene castor oil is dissolved. Subsequently paclitaxel at a content of 101.09% (593.5 mg) in 100 ml of this solution is added and stirred until the solution becomes clear.

The solution is divided into two portions, one of which is filled without cation exchanger treatment at 5 ml into sterilized punctable bottles via 0.2 μm filters, placed under a stream of nitrogen and closed with a sterilized stopper for injection solutions.

The other portion of the solution is treated with the cation exchanger (0.1 g/100 g solution). The solution is shaken for 16 hours. This solution is filled via 0.2 μm filters at 5 ml each into sterilized punctable bottles, placed under a stream of nitrogen and closed with a sterilized stopper for injection solutions. A portion of the samples is used for the initial analysis. The remaining punctable bottles are stored for two weeks at 60° C. and subsequently analyzed.

### Example 3 and 4

Polyoxyethylene castor oil (25.44 g) is dissolved in ethanol (78.06 g) and stirred until all of the polyoxyethylene castor oil is dissolved. Subsequently paclitaxel at a content of 101.09% (593.5 mg) in 100 ml of this solution is added and stirred until the solution becomes clear.

The further process is carried out as described in Example 1 and 2.

### Example 5 and 6

Polyoxyethylene castor oil (38.16 g) is dissolved in ethanol (68.30 g) and stirred until all of the polyoxyethylene castor oil is dissolved. Subsequently paclitaxel at a content of 101.09% (593.5 mg) in 100 ml of this solution is added and stirred until the solution becomes clear.

The further process is carried out as described in Example 1 and 2.

### Example 7 and 8

Polysorbate 80 (60 ml) is added to ethanol (60 ml) and stirred until a homogeneous solution is formed. Paclitaxel at a content of 101.09% (593.5 mg) in 100 ml of this solution is subsequently added and stirred until the solution has become clear.

The further process is carried out as described in Example 1 and 2.

### Example 9 and 10

Paclitaxel at a content of 101.09% (593.5 mg) in 100 ml of ethanol is stirred until the solution has become clear.

The further process is carried out as described in Example 1 and 2.

### Example 11 and 12

Polyoxyethylene castor oil (56.52 g) is dissolved in ethanol (43.90 g) and stirred until all of the polyoxyethylene castor oil is dissolved. Subsequently, paclitaxel at a content of 101.09% (593.5 mg) in 100 ml of this solution is added and stirred until the solution has become clear.

### Example 14

Polyoxyethylene castor oil (56.52 g) is dissolved in ethanol (43.90 g) and the solution is stirred until all of the polyoxyethylene castor oil is dissolved. Subsequently, paclitaxel at a content of 101.09% (593.5 mg) and anhydrous citric acid (200 mg) in 100 ml of this solution is added and stirred until the solution has become clear.

| | Example | | | | | | | | | | | | | |
|---|---|---|---|---|---|---|---|---|---|---|---|---|---|---|
| | 1 | | 2 | | 3 | | 4 | | 5 | | 6 | | 7 | |
| | Description Paclitaxel 6 mg/ml | | | | | | | | | | | | | |
| | Polyoxy- | | | | Polyoxy- | | | | Polyoxy- | | | | | |
| | ethylene | | Polyoxy- | | ethylene | | Polyoxy- | | ethylene | | Polyoxy- | | | |
| | castor oil: | | ethylene | | castor oil: | | ethylene | | castor oil: | | ethylene | | | |
| | ethanol = | | castor oil: | | ethanol = | | castor oil: | | ethanol = | | castor oil: | | Polysorbate 80: | |
| | 10:90 | | ethanol = | | 20:80 | | ethanol = | | 30:70 | | ethanol = | | ethanol = 50:50 | |
| | treated | | 10:90 | | treated | | 20:80 | | treated | | 30:70 | | treated | |
| | according to | | without | | according to | | without | | according to | | without | | according to | |
| | the invention | | treatment | | the invention | | treatment | | the invention | | treatment | | the invention | |
| | Storage condition | | | | | | | | | | | | | |
| | ini- | 60° C./ | ini- | 60° C./ | ini- | 60° C./ | ini- | 60° C./ | ini- | 60° C./2 | ini- | 60° C./ | ini- | 60° C./ |
| | tial | 2 weeks | tial | 2 weeks | tial | 2 weeks | tial | 2 weeks | tial | weeks | tial | 2 weeks | tial | 2 weeks |
| HPLC content [%] | 101.6 | 99.1 | 101.0 | 88.7 | 105.2 | 103.7 | 102.9 | 90.1 | 100.26 | 99.80 | 98.93 | 81.20 | 100.1 | 80.28 |
| Impurities [%] | | | | | | | | | | | | | | |
| 10-Deacetyl- | <0.1 | <0.1 | <0.1 | <0.1 | <0.1 | <0.1 | <0.1 | <0.1 | <0.1 | <0.1 | <0.1 | <0.1 | <0.1 | <0.1 |
| baccatin III | | | | | | | | | | | | | | |
| Baccatin III | <0.1 | <0.1 | <0.1 | 3.9 | <0.1 | <0.1 | <0.1 | 4.9 | <0.1 | <0.1 | <0.1 | 6.7 | <0.1 | 7.0 |
| (2R,3S)-N | <0.1 | <0.1 | <0.1 | 3.3 | <0.1 | <0.1 | <0.1 | 4.1 | <0.1 | <0.1 | <0.1 | 5.7 | <0.1 | 5.4 |
| benzoyl phenyl | | | | | | | | | | | | | | |
| isoserine ethyl | | | | | | | | | | | | | | |
| ester | | | | | | | | | | | | | | |
| 10-Deacetyl | <0.1 | <0.1 | <0.1 | 0.3 | <0.1 | <0.1 | <0.1 | 0.3 | <0.1 | <0.1 | <0.1 | 0.4 | <0.1 | 0.5 |
| paclitaxel | | | | | | | | | | | | | | |
| Cephalomannines | <0.1 | <0.1 | <0.1 | 0.1 | <0.1 | <0.1 | <0.1 | <0.1 | <0.1 | <0.1 | <0.1 | 0.1 | <0.1 | <0.1 |
| 10-Deacetyl-7- | <0.1 | <0.1 | <0.1 | 0.2 | <0.1 | <0.1 | <0.1 | 0.2 | <0.1 | <0.1 | <0.1 | 0.1 | <0.1 | 0.2 |
| epipaclitaxel | | | | | | | | | | | | | | |
| 7-Epipaclitaxel | 0.4 | 0.5 | 0.6 | 3.2 | 0.2 | 0.3 | 0.8 | 3.4 | 0.2 | 0.2 | 0.5 | 4.0 | 0.2 | 6.1 |
| Total impurities | 0.6 | 0.5 | 0.8 | 11.0 | 0.2 | 0.3 | 0.8 | 13.1 | 0.3 | 0.5 | 0.7 | 17.2 | 0.2 | 19.7 |

| | Example | | | | | | | | | | | | | |
|---|---|---|---|---|---|---|---|---|---|---|---|---|---|---|
| | 8 | | 9 | | 10 | | 11 | | 12 | | 13 | | 14 | |
| | Description Paclitaxel 6 mg/ml | | | | | | | | | | | | | |
| | | | | | | | Polyoxyethylene | | | | | | | |
| | | | | | | | castor oil: | | Polyoxyethylene | | | | Polyoxyethylene | |
| | Polysorbate 80: | | | | | | ethanol = | | castor oil: | | | | castor oil: | |
| | ethanol = | | Ethanol | | | | 50:50 | | ethanol = | | Taxol ® | | ethanol = | |
| | 50:50 | | treated | | Ethanol | | treated | | 50:50 | | Bristol | | 50:50 | |
| | without | | according to | | without | | according to | | without | | Meyers | | with | |
| | treatment | | the invention | | treatment | | the invention | | treatment | | Squibb | | citric acid | |
| | Storage condition | | | | | | | | | | | | | |
| | ini- | 60° C./ | ini- | 60° C./ | ini- | 60° C./ | ini- | 60° C./ | ini- | 60° C./ | ini- | 60° C./ | ini- | 60° C./ |
| | tial | 2 weeks | tial | 2 weeks | tial | 2 weeks | tial | 2 weeks | tial | 2 weeks | tial | 2 weeks | tial | 2 weeks |
| HPLC content [%] | 100.19 | 45.12 | 101.0 | 100.6 | 104.2 | 67.9 | 102.9 | 99.9 | 99.5 | 86.4 | 100.8 | 100.0 | 100.5 | 98.90 |
| Impurities [%] | | | | | | | | | | | | | | |
| 10-Deacetyl- | <0.1 | 0.2 | <0.1 | <0.1 | <0.1 | <0.1 | <0.1 | <0.1 | <0.1 | <0.1 | <0.1 | <0.1 | <0.1 | <0.1 |
| baccatin III | | | | | | | | | | | | | | |
| Baccatin III | 0.1 | 16.2 | <0.1 | <0.1 | <0.1 | 12.3 | <0.1 | <0.1 | <0.1 | 4.2 | 0.1 | 0.4 | <0.1 | <0.1 |
| (2R,3S)-N | <0.1 | 14.5 | <0.1 | <0.1 | <0.1 | 11.5 | <0.1 | <0.1 | <0.1 | 3.2 | <0.1 | 0.4 | <0.1 | <0.1 |
| benzoyl phenyl | | | | | | | | | | | | | | |
| isoserine ethyl | | | | | | | | | | | | | | |
| ester | | | | | | | | | | | | | | |
| 10-Deacetyl | <0.1 | 0.8 | <0.1 | 0.4 | <0.1 | 0.4 | <0.1 | <0.1 | <0.1 | 0.8 | 0.2 | 0.5 | <0.1 | 0.3 |
| paclitaxel | | | | | | | | | | | | | | |
| Cephalomannines | <0.1 | <0.1 | <0.1 | <0.1 | <0.1 | 0.1 | 0.1 | 0.1 | 0.1 | 0.2 | 0.2 | 0.3 | 0.2 | 0.2 |
| 10-Deacetyl-7- | <0.1 | 0.8 | <0.1 | <0.1 | <0.1 | 0.3 | <0.1 | <0.1 | <0.1 | 0.1 | <0.1 | <0.1 | <0.1 | <0.1 |
| epipaclitaxel | | | | | | | | | | | | | | |
| 7-Epipaclitaxel | 0.7 | 15.7 | 0.1 | 0.5 | 0.1 | 7.6 | <0.1 | 0.1 | 0.1 | 2.9 | 0.5 | 0.7 | 0.1 | 0.2 |
| Total impurities | 0.8 | 51.1 | 0.3 | 2.0 | 0.2 | 33.3 | 0.2 | 0.7 | 0.7 | 12.4 | 1.2 | 2.5 | 0.4 | 2.0 |

The examples explain the invention without limiting it.

1-10. (canceled)

11. Method for the production of a stable injectable formulation of poorly soluble antineoplastic agents, wherein a formulation comprising the antineoplastic agent and a solvent and/or a solvent system, which optionally contains a solubilizing agent, is treated with a cation exchanger.

12. Method as claimed in claim 11, wherein the antineoplastic agent is paclitaxel, camptothecine or teniposide.

13. Method as claimed in claim 12, wherein the antineoplastic agent is paclitaxel.

14. Method as claimed in claim 11, wherein the content of the active agent in the solution is 1-10 mg/ml.

15. Method as claimed in claim 14, wherein the content of the active agent paclitaxel is 4-8 mg/ml.

16. Method as claimed in claim 15, wherein the content of the active agent paclitaxel is 6 mg/ml.

17. Method as claimed in claim 11, wherein as the solvent or solvent system with solubilizing agent are utilized ethanol, ethanol/polyoxyethylene castor oil, ethanol/polysorbate and ethanol/polyethylene glycol.

18. Method as claimed in claim 17, wherein the content of ethanol in the solvent system ethanol/polyoxyethylene castor oil is 10-90 parts.

19. Method as claimed in claim 17, wherein the content of ethanol in the solvent system ethanol/polysorbate is 40-60 parts.

20. Method as claimed in claim 11, wherein as the cation exchanger an ion exchanger containing sulfonic acid groups or carboxylate groups is employed.

21. Method as claimed in claim 11, wherein the quantity of the cation exchanger is 0.01-10% of the total batch.
